(19) United States
(12) Patent Application Publication
(10) Pub. No.: US 2022/0169637 A1
(43) Pub. Date: Jun. 2, 2022

(54) SOLID FORMS OF ENCEQUIDAR MESYLATE AND PROCESSES THEREOF

(71) Applicant: DR. REDDY'S LABORATORIES LIMITED, Hyderabad (IN)

(72) Inventors: Srinivas ORUGANTI, Hyderabad (IN); Vishnu Vardhana Vema Reddy EDA, Hyderabad (IN); Saikat SEN, Kolkata (IN); Arijit MUKHERJEE, Basirhat (IN); Satyanarayana THIRUNAHARI, Hyderabad (IN)

(21) Appl. No.: 17/442,297

(22) PCT Filed: Mar. 24, 2020
(86) PCT No.: PCT/IB2020/052732

§ 371 Date: Sep. 23, 2021

(30) Foreign Application Priority Data
Mar. 25, 2019 (IN) 201941011597
Sep. 13, 2019 (IN) 201941036910
Oct. 1, 2019 (IN) 201941039699

(51) Int. Cl. C07D 405/14

(52) U.S. Cl. CPC C07D 405/14; C07B 2200/13

(57) ABSTRACT

Aspects of the present application relate to solid forms of Encequidar, its mesylate salt and pharmaceutical compositions thereof. Specific aspects relate to the crystalline Form E1 of Encequidar, crystalline Form EM1, crystalline Form EM2 and crystalline Form EM3 of Encequidar mesylate. Further aspects relate to processes for the preparation of solid forms of Encequidar and its mesylate salt.

### INTRODUCTION

Aspects of the present application relate to solid forms of Encequidar, its mesylate salt and pharmaceutical compositions thereof. Specific aspects relate to the crystalline forms of Encequidar, mesylate salt thereof and processes for their preparation.

Encequidar is the adopted name of compound developed by Hanmi pharma having a chemical name: N-[2-(2-{4-[2-(6,7-Dimethoxy-3,4-dihydro-2(1H)isoquinolinyl)ethyl]phenyl}-2H-tetrazol-5-yl)-4,5-dimethoxyphenyl]-4-oxo-4H-chromene-2-carboxamide and the structure as below.

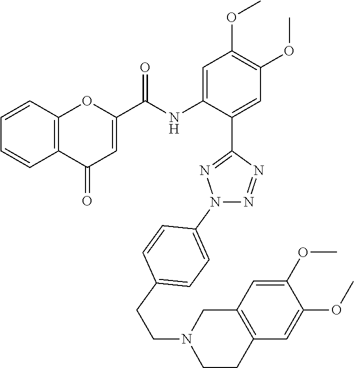 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]

Encequidar is P-glycoprotein pump inhibitor, which can facilitate oral absorption of traditional cytotoxics such as Paclitaxel, Docetaxel, Topotecan, Irinotecan and Eribulin for improved patient tolerability and efficacy as compared to IV administration of the same cytotoxics.

U.S. Pat. No. 7,625,926 B2 first discloses Encequidar, its mesylate salt, preparative process, pharmaceutical composition and their use for inhibiting activity of P-glycoprotein.

Further, U.S. Pat. No. 9,283,218 B2 discloses a crystalline form of mesylate salt of Encequidar which is characterized through X-ray power diffraction pattern. However, U.S. Pat. No. 9,283,218 B2 discloses neither the preparation nor the stability and viability of said polymorph in a pharmaceutical dosage form.

None of these arts disclose an amenable and/or scalable solid form of Encequidar mesylate that can be formulated as drug product. Hence, there remains a need for alternate solid forms of Encequidar mesylate which can overcome the disadvantages of the prior art and their preparation in a more cost effective and industrially viable manner.

### SUMMARY

In an aspect, the present application provides a crystalline Form EM1 of Encequidar mesylate, characterized by a PXRD pattern comprising the peaks at about 8.78, 10.68, 23.84 and 25.55±0.2° 2θ.

In another aspect, the present application provides a crystalline Form EM2 of Encequidar mesylate, characterized by a PXRD pattern comprising the peaks at about 16.28, 21.93 and 24.77±0.2° 2θ.

In another aspect, the present application provides a crystalline Form EM3 of Encequidar mesylate, characterized by a PXRD pattern comprising the peaks at about 22.85 and 25.91±0.2° 2θ.

In another aspect, the present application provides a crystalline Form E1 of Encequidar, characterized by a PXRD pattern comprising the peaks at about 9.79, 11.00 and 24.07±0.2° 2θ.

In another aspect, the present application provides a process for the preparation of crystalline Form EM1 of Encequidar mesylate, comprising the steps of combining Encequidar mesylate with an inert solvent and isolating crystalline Form EM1.

In another aspect, the present application provides a process for the preparation of crystalline Form EM1 of Encequidar mesylate, comprising the step of treating amorphous Encequidar mesylate with a suitable inert solvent, optionally in the presence of seed crystals of Form EM1.

In another aspect, the present application provides a process for the preparation of crystalline Form EM2 of Encequidar mesylate, comprising the steps of combining Encequidar mesylate with nitromethane and isolating crystalline Form EM2.

In another aspect, the present application provides a process for the preparation of crystalline Form EM3 of Encequidar mesylate, comprising the step of drying crystalline Form EM2 of Encequidar mesylate.

In another aspect, the present application provides a process for the preparation of crystalline Form E1 of Encequidar, comprising the steps of combining Encequidar with an inert solvent and isolating crystalline Form E1.

In another aspect, the present application provides a process comprising the step of converting crystalline Form E1 of Encequidar to salt form of Encequidar.

In another aspect, the present application provides pharmaceutical compositions comprising a crystalline form of Encequidar mesylate, selected from the group consisting of EM1, EM2, EM3 and mixtures thereof together with at least one pharmaceutically acceptable excipient.

### BRIEF DESCRIPTION OF THE DRAWING

FIG. 1 is an illustrative X-ray powder diffraction pattern of crystalline Form E1 of Encequidar, prepared by the method of Example No 1.

FIG. 2 is an illustrative X-ray powder diffraction pattern of crystalline Form EM1 of Encequidar mesylate, prepared by the method of Example No 2.

FIG. 3 is an illustrative X-ray powder diffraction pattern of crystalline Form EM2 of Encequidar mesylate, prepared by the method of Example No 3.

FIG. 4 is an illustrative X-ray powder diffraction pattern of crystalline Form EM3 of Encequidar mesylate, prepared by the method of Example No 4.

FIG. 5 is an illustrative X-ray powder diffraction pattern of crystalline Form EM1 of Encequidar mesylate subjected to stress conditions.

### DETAILED DESCRIPTION

In an aspect, the present application provides a crystalline Form EM1 of Encequidar mesylate, characterized by a PXRD pattern comprising the peaks at about 8.78, 10.68, 23.84 and 25.55±0.2° 2θ.

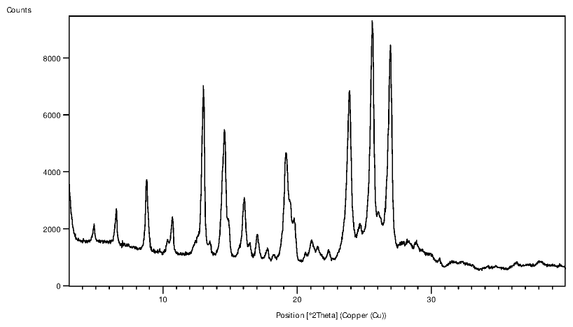

In embodiments, the application provides crystalline Form EM1 of Encequidar mesylate, characterized by a PXRD pattern of FIG. 2.

In embodiments, the present application provides a stable crystalline Form EM1 of Encequidar mesylate which is stable under stress conditions. In embodiments, crystalline Form EM1 of Encequidar mesylate is stable under all ICH storage and packing conditions.

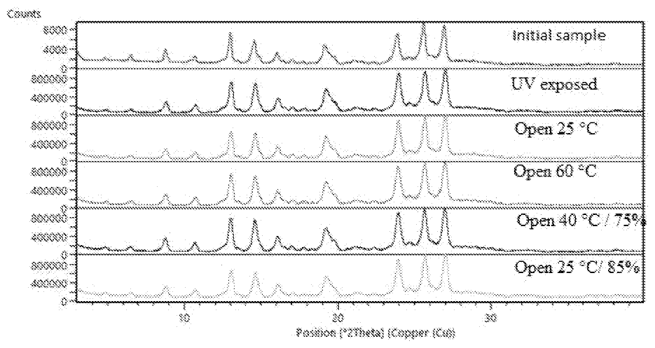

In an embodiment, the open sample of Form EM1 is stable at 25° C. for at least 24 hours. In an embodiment, the open sample of Form EM1 is stable under heating in air tray drier at 60° C. for at least 24 hours. In an embodiment, the open sample of Form EM1 is stable at 40° C. and 75% RH condition for at least 24 hours. In an embodiment, the open sample of Form EM1 is stable at 25° C. and 85% RH for at least 24 hours. In an embodiment, the open sample of Form EM1 is stable under UV exposure for at least 16 hours. No significant change was observed in weight or moisture content of the samples and the X-ray diffraction patterns, as depicted in FIG. 5.

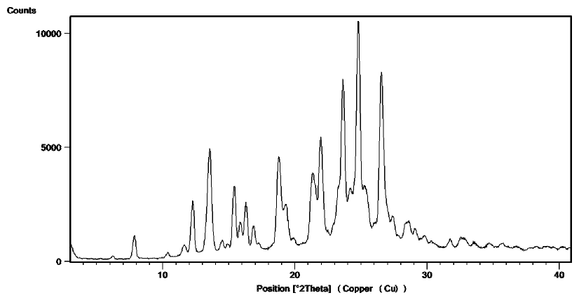

In another aspect, the present application provides a crystalline Form EM2 of Encequidar mesylate, characterized by a PXRD pattern comprising the peaks at about 16.28, 21.93 and 24.77±0.2° 2θ. In embodiments, the crystalline Form EM2 of Encequidar mesylate is characterized by one or more additional peaks at about 12.24, 23.62 and 26.52° 2θ. In embodiments, the application provides crystalline Form EM2 of Encequidar mesylate, characterized by a PXRD pattern of FIG. 3.

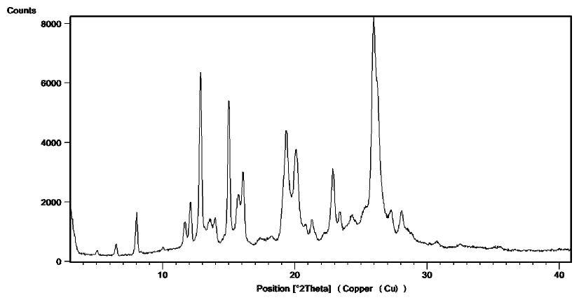

In another aspect, the present application provides a crystalline Form EM3 of Encequidar mesylate, characterized by a PXRD pattern comprising the peaks at about 22.85 and 25.91±0.2° 2θ. In embodiments, the crystalline Form EM3 of Encequidar mesylate is characterized by one or more additional peaks at about 14.98 and 20.06° 2θ. In embodiments, the application provides crystalline Form EM3 of Encequidar mesylate, characterized by a PXRD pattern of FIG. 4.

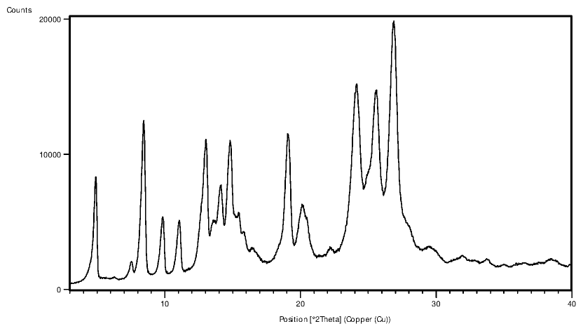

In another aspect, the present application provides a crystalline Form E1 of Encequidar, characterized by a PXRD pattern comprising the peaks at about 9.79, 11.00 and 24.07±0.2° 2θ. In embodiments, the crystalline Form E1 of Encequidar is characterized by one or more additional peaks at about 19.03, 25.50 and 26.83° 2θ. In embodiments, the application provides crystalline Form E1 of Encequidar, characterized by a PXRD pattern of FIG. 1.

In another aspect, the present application provides a process for the preparation of crystalline Form EM1 of Encequidar mesylate, comprising the steps of combining Encequidar mesylate with an inert solvent and isolating crystalline Form EM1.

In embodiments, Encequidar mesylate used in this aspect may be obtained by any methods known in the art or procedures described or exemplified in the present application, comprising the reaction of Encequidar with methane sulphonic acid under suitable conditions to form mesylate salt of Encequidar.

In embodiments, combining Encequidar mesylate with an inert solvent may be carried out by combining the reaction mixture containing Encequidar and methane sulfonic acid with an inert solvent.

In embodiments, combining Encequidar mesylate may be carried out through the formation of a homogeneous solution or a heterogeneous mixture containing Encequidar mesylate in inert solvent, under suitable temperature at about 0° C. to reflux temperature of the inert solvent or mixtures thereof.

In embodiments, combining Encequidar mesylate may be carried out by dissolving Encequidar mesylate in inert solvent. In embodiments, the solvent from solution containing Encequidar mesylate may be optionally removed using suitable methods known in the art or according to procedures described in the present application. In embodiments, the solvent from solution containing Encequidar mesylate may be removed completely or partially.

In embodiments, the solvent from the solution containing Encequidar mesylate may be removed through crystallization either by cooling the solution or by addition of anti-solvent, followed by separation of the solids by filtration or decantation.

In embodiments, the solvent from solution containing Encequidar mesylate may be removed by evaporating or sublimating the solvent, optionally under reduced pressure at about 0° C. to reflux temperature of inert solvent or mixtures thereof.

In embodiments, the product obtained after the removal of solvent may optionally be again combined with same or different inert solvent. In embodiments, combining the product containing Encequidar mesylate may be carried out through the formation of a homogeneous solution or a heterogeneous mixture in inert solvent, under suitable temperature at about 0° C. to reflux temperature of the inert solvent or mixtures thereof. In embodiments, the process of combining this product containing Encequidar mesylate with the inert solvent may be repeated at least one time or more to obtain crystalline Form EM1 of Encequidar mesylate.

In embodiments, combining the product with inert solvent may be carried by dissolving the product in inert solvent at suitable temperature of about 0° C. to reflux temperature to form a solution containing Encequidar mesylate.

In embodiments, combining the product with inert solvent may be carried out by suspending the product in inert solvent at suitable temperature of about 0° C. to reflux temperature to form a slurry or suspension containing Encequidar mesylate.

In embodiments, combining the product with inert solvent may be carried out for sufficient time to obtain crystalline form EM1 of Encequidar mesylate. In preferred embodiments, combining the product with inert solvent may be carried out for at least one hour or longer to obtain crystalline form EM1 of Encequidar mesylate.

In embodiments, combining the product with inert solvent may be carried out at suitable temperature to obtain crystalline form EM1 of Encequidar mesylate.

In preferred embodiments, suspending the product in inert solvent may be carried out at suitable temperature of about 0° C. to reflux temperature to obtain crystalline form EM1 of Encequidar mesylate.

In embodiments, inert solvent of this aspect may be selected from the group consisting of water, ketone solvent, alcohol solvent, nitrile solvent, ether solvent, hydrocarbon solvent, halohydrocarbon solvent, polar aprotic solvents, ester solvents, and mixtures thereof. In embodiments, ketone solvent such as Acetone, Methyl ethyl ketone, Methyl isobutyl ketone; alcohol solvent such as methanol, ethanol, 1-propanol, 2-propanol; nitrile solvent such as acetonitrile, propionitrile; ether solvents such as diethylether, diisopropyl ether, methyl tert.butyl ether, tetrahydrofuran, 1,4-dioxane, Anisole; hydrocarbon solvents such as hexane, heptane, cyclohexane, petroleum ether; halohydrocarbon solvents such as dichloromethane, chloroform, carbon tetrachloride; ester solvents such as ethyl acetate, isopropyl acetate, methyl acetate; polar aprotic solvents such as dimethyl formamide, dimethyl sulfoxide and mixtures thereof. In preferred embodiments, the inert solvent may be selected from the group consisting of water, ketone solvent, alcohol solvent, nitrile solvent and mixtures thereof.

In embodiments, the crystalline form EM1 of Encequidar mesylate may be isolated by separating the solids from the solvent through suitable techniques known in the art such as filtration, decantation and the like. The isolated solids may be dried under suitable drying conditions such as aerial drying, drying under vacuum or inert gas at a suitable temperature of about 25° C. or above.

In embodiments, the crystalline Form EM1 of Encequidar mesylate obtained by the process of this aspect may be characterized by a PXRD pattern comprising the peaks at about 8.78, 10.68, 23.84 and 25.55±0.2° 2θ. In embodiments, crystalline Form EM1 may be characterized by a PXRD pattern of FIG. 2.

In another aspect, the present application provides a process for the preparation of crystalline Form EM1 of Encequidar mesylate, comprising the step of treating amorphous Encequidar mesylate with a suitable inert solvent, optionally in the presence of seed crystals of Form EM1.

In embodiments, treating amorphous Encequidar mesylate with the solvent may be carried out by suspending or dissolving amorphous Encequidar mesylate in the solvent.

In an embodiment, the amorphous Encequidar mesylate may be suspended in a solvent or mixture of solvents, optionally in the presence of seed crystals of Form EM1.

In an embodiment, the amorphous Encequidar mesylate may be dissolved in a solvent or mixture of solvents and the saturated solution may be crystallized in the presence of seed crystals of Form EM1. In embodiments, crystallization of solution may be carried out through suitable method by cooling or evaporating the solution and/or by contacting with an anti-solvent.

In embodiments, inert solvent of this aspect may be selected from the group consisting of water, ketone solvent, alcohol solvent, nitrile solvent, ether solvent, hydrocarbon solvent, halohydrocarbon solvent, polar aprotic solvents, ester solvents, and mixtures thereof. In embodiments, ketone solvent such as Acetone, Methyl ethyl ketone, Methyl isobutyl ketone; alcohol solvent such as methanol, ethanol, 1-propanol, 2-propanol; nitrile solvent such as acetonitrile, propionitrile; ether solvents such as diethylether, diisopropyl ether, methyl tert.butyl ether, tetrahydrofuran, 1,4-dioxane, Anisole; hydrocarbons solvents such as hexane, heptane, cyclohexane, petroleum ether; halohydrocarbon solvents such as dichloromethane, chloroform, carbon tetrachloride; ester solvents such as ethyl acetate, isopropyl acetate, methyl acetate; polar aprotic solvents such as dimethyl formamide, dimethyl sulfoxide and mixtures thereof. In preferred embodiments, the inert solvent may be selected from the group consisting of water, ketone solvent, alcohol solvent, nitrile solvent, ether solvent and mixtures thereof.

Anti-solvent is solvent in which Encequidar mesylate is either insoluble or low soluble.

In embodiments, the amount of seed crystals of Form EM1 may be between 0.1% and 1% of the amorphous Encequidar mesylate.

In embodiments, treating amorphous Encequidar mesylate with the solvent may be carried out at suitable temperature of about 0° C. to reflux temperature of the solvent.

In embodiments, treating amorphous Encequidar mesylate with the solvent may be carried for sufficient time to complete the formation of Encequidar mesylate, for about 1 hour or more.

In embodiments, the crystalline form EM1 of Encequidar mesylate may be isolated by separating the solids from the solvent through suitable techniques known in the art such as filtration, decantation and the like. The isolated solids may be dried under suitable drying conditions such as aerial drying, drying under vacuum or inert gas at a suitable temperature of about 25° C. or above.

In embodiments, the crystalline Form EM1 of Encequidar mesylate obtained by the process of this aspect may be characterized by a PXRD pattern comprising the peaks at about 8.78, 10.68, 23.84 and 25.55±0.2° 2θ. In embodiments, crystalline Form EM1 may be characterized by a PXRD pattern of FIG. 2.

In another aspect, the present application provides a process for the preparation of crystalline Form EM2 of Encequidar mesylate, comprising the steps of combining Encequidar mesylate with nitromethane and isolating crystalline Form EM2.

In embodiments, Encequidar mesylate used in this aspect may be obtained by any methods known in the art or procedures described or exemplified in the present application, comprising the reaction of Encequidar with methane sulphonic acid under suitable conditions to form mesylate salt of Encequidar.

In embodiments, combining Encequidar mesylate with an inert solvent may be carried out by combining the reaction mixture containing Encequidar and methane sulfonic acid with nitromethane.

In embodiments, combining Encequidar mesylate may be carried out through the formation of a homogeneous solution or a heterogeneous mixture containing Encequidar mesylate in nitromethane, under suitable temperature at about 0° C. to reflux temperature of solvent or mixtures thereof.

In embodiments, combining Encequidar mesylate with nitromethane may be carried out by dissolving Encequidar mesylate in nitromethane, optionally by heating. In embodiments, the solution containing Encequidar mesylate in nitromethane may be held for sufficient time and suitable temperature to isolate crystals of Form EM2. In embodiments, the solution may be held overnight to isolate Form EM2. In embodiments, the solution may be held in closed condition or in open condition or in combination thereof. In embodiments, the solution may be held at about 0° C. or above.

In embodiments, the solvent from solution containing Encequidar mesylate may be optionally removed using suitable methods known in the art or according to procedures described in the present application. In embodiments, the solvent from solution containing Encequidar mesylate may be removed completely or partially to obtain crystalline Form EM2 of Encequidar mesylate.

In embodiments, the solvent from the solution containing combining Encequidar mesylate may be removed through crystallization either by cooling the solution or by addition of anti-solvent, followed by separation of the solids by filtration or decantation.

In embodiments, the solvent from solution containing Encequidar mesylate may be removed by evaporating or sublimating the solvent, optionally under reduced pressure at about 0° C. to reflux temperature of inert solvent or mixtures thereof.

In embodiments, the crystalline form EM2 of Encequidar mesylate may be isolated by separating the solids from the solvent through suitable techniques known in the art such as filtration, decantation and the like. The isolated solids may be dried under suitable drying conditions such as aerial drying, drying under vacuum or inert gas at a suitable temperature of about 25° C. or above.

In embodiments, the crystalline Form EM2 of Encequidar mesylate obtained by the process of this aspect may be characterized by a PXRD pattern comprising the peaks at about 16.28, 21.93 and 24.77±0.2° 2θ. In embodiments, crystalline Form EM2 may be characterized by a PXRD pattern of FIG. 3.

In another aspect, the present application provides a process for the preparation of crystalline Form EM3 of Encequidar mesylate, comprising the step of drying crystalline Form EM2 of Encequidar mesylate.

In embodiments, Encequidar mesylate or its crystalline form EM2 used in this aspect may be obtained by any methods known in the art or procedures described or exemplified in the present application, comprising the reaction of Encequidar with methane sulphonic acid under suitable conditions to form mesylate salt of Encequidar. Crystalline form EM2 of Encequidar mesylate may be obtained by combining Encequidar mesylate with nitromethane and isolating crystalline form EM2.

In embodiments, crystalline form EM2 of Encequidar mesylate may be dried under suitable drying conditions such as aerial drying, drying under vacuum or inert gas at a suitable temperature of about 25° C. or above.

In embodiments, crystalline form EM2 may be dried in an open container under at suitable temperature for sufficient time to convert into crystalline form EM3. In embodiments, crystalline form EM2 may be dried under controlled humidity conditions.

In embodiments, crystalline form EM2 may be dried optionally under heating conditions. In embodiments, the crystalline form EM2 may be heated to a temperature of about 25° C. or above.

In embodiments, the crystalline form EM2 may be dried optionally under reduced pressure conditions.

In embodiments, the crystalline form EM2 may be dried optionally under inert gas atmosphere such as nitrogen.

In embodiments, the crystalline form EM2 may be dried for at least one hour or longer.

In embodiments, the crystalline Form EM3 of Encequidar mesylate obtained by the process of this aspect may be characterized by a PXRD pattern comprising the peaks at about 22.85, 25.91±0.2° 2θ. In embodiments, crystalline Form EM3 may be characterized by a PXRD pattern of FIG. 4.

In another aspect, the present application provides a process for the preparation of crystalline Form E1 of Encequidar, comprising the steps of providing a mixture of Encequidar and an inert solvent and isolating crystalline Form E1.

In embodiments, Encequidar used in this aspect may be obtained by any methods known in the art or procedures described or exemplified in the present application, such as a process comprising the step of reacting 2-(2-(4-(2-(6,7-dimethoxy-3,4-dihydroisoquinolin-2(1H)-yl)ethyl)phenyl)-2H-tetrazol-5-yl)-4,5-dimethoxyaniline with 4-oxo-4H-chromene-2-carboxylic acid or any activated 4-oxo-4H-chromene-2-carboxylic acid.

In embodiments, the reaction mixture containing Encequidar in an inert solvent may be taken directly or Encequidar may be combined with an inert solvent. In embodiments, the reaction mixture may be a homogeneous solution or a heterogeneous mixture.

In embodiments, combining Encequidar may be carried out through the formation of a homogeneous solution or a heterogeneous mixture in inert solvent, under suitable temperature at about 0° C. to reflux temperature of the inert solvent or mixtures thereof.

In embodiments, combining Encequidar may be carried out by dissolving Encequidar in inert solvent. In embodiments, the solvent from solution containing Encequidar may be optionally removed using suitable methods known in the art or according to procedures described in the present application. In embodiments, the solvent from solution containing Encequidar may be removed completely or partially.

In embodiments, the solvent from the solution containing Encequidar may be removed through crystallization either by cooling the solution or by addition of anti-solvent, followed by separation of the solids by filtration or decantation.

In embodiments, the solvent from solution containing Encequidar may be removed by evaporating or sublimating the solvent, optionally under reduced pressure at about 0° C. to reflux temperature of inert solvent or mixtures thereof.

In embodiments, the product obtained may be optionally again combined with same or different inert solvent. In embodiments, combining the product containing Encequidar may be carried out through the formation of a homogeneous solution or a heterogeneous mixture in inert solvent, under suitable temperature at about 0° C. to reflux temperature of the inert solvent or mixtures thereof.

In embodiments, combining the product with inert solvent may be carried out by dissolving the product in inert solvent at suitable temperature of about 0° C. to reflux temperature to form a solution containing Encequidar.

In embodiments, combining the product with inert solvent may be carried out by suspending the product in inert solvent at suitable temperature of about 0° C. to reflux temperature to form a slurry or suspension containing Encequidar.

In embodiments, combining the product with inert solvent may be carried out for sufficient time to obtain crystalline form E1 of Encequidar. In preferred embodiments, combining the product with inert solvent may be carried out for at least one hour or longer to obtain crystalline form E1 of Encequidar.

In embodiments, combining the product with inert solvent may be carried out at suitable temperature to obtain crystalline form E1 of Encequidar.

In preferred embodiments, suspending the product in inert solvent may be carried out at suitable temperature of about 0° C. to reflux temperature to obtain crystalline form E1 of Encequidar.

In embodiments, inert solvent of this aspect may be selected from the group consisting of water, ketone solvent, alcohol solvent, nitrile solvent, ether solvent, hydrocarbon solvent, halohydrocarbon solvent, ester solvents, polar aprotic solvents and mixtures thereof. In embodiments, ketone solvent such as Acetone, Methyl ethyl ketone, Methyl isobutyl ketone; alcohol solvent such as methanol, ethanol, 1-propanol, 2-propanol; nitrile solvent such as acetonitrile, propionitrile; ether solvents such as diethylether, diisopropyl ether, methyl tert.butyl ether, tetrahydrofuran, 1,4-dioxane; hydrocarbon solvents such as hexane, heptane, cyclohexane, petroleum ether; halohydrocarbon solvents such as dichloromethane, chloroform, carbon tetrachloride; ester solvents such as ethyl acetate, isopropyl acetate, methyl acetate; polar aprotic solvents such as dimethyl formamide, dimethyl acetamide, dimethyl sulfoxide, and mixtures thereof. In preferred embodiments, the inert solvent may be selected from the group consisting of water, ketone solvent, alcohol solvent, nitrile solvent and mixtures thereof.

In embodiments, the crystalline form E1 of Encequidar may be isolated by separating the solids from the solvent through suitable techniques known in the art such as filtration, decantation and the like. The isolated solids may be dried under suitable drying conditions such as aerial drying, drying under vacuum or inert gas at a suitable temperature of about 25° C. or above.

In embodiments, the crystalline Form E1 of Encequidar obtained by the process of this aspect may be characterized by a PXRD pattern comprising the peaks at about 9.79, 11.00 and 24.07±0.2° 2θ. In embodiments, the crystalline Form E1 of Encequidar is characterized by one or more additional peaks at about 19.03, 25.50 and 26.83° 2θ. In embodiments, crystalline Form EM1 may be characterized by a PXRD pattern of FIG. 1.

In another aspect, the present application provides a process comprising the step of converting crystalline Form E1 of Encequidar to salt form of Encequidar.

In embodiments, the process comprising the step of converting crystalline Form E1 of Encequidar to mesylate salt of Encequidar. In embodiments, converting crystalline Form E1 of Encequidar to mesylate salt of Encequidar may be carried out by treating crystalline Form E1 of Encequidar with methane sulfonic acid, optionally in the presence of an inert solvent.

In embodiments, the crystalline Form E1 of Encequidar is useful to prepare Encequidar with desired chemical purity before converting it into a suitable salt form.

In embodiments, the crystalline Form E1 of Encequidar is useful as an intermediate in the preparation of corresponding salt form. In embodiments, the crystalline Form E1 of Encequidar may be used as an intermediate in the preparation of mesylate salt of Encequidar.

In another aspect, the present application provides crystalline form of Encequidar mesylate selected from the group consisting of EM1, EM2, EM3 and mixtures thereof according to instant application and pharmaceutical compositions thereof, wherein the chemical purity of Encequidar mesylate may be more than 99% by HPLC or more than 99.5% by HPLC or more than 99.9% by HPLC.

In another aspect, the present application provides pharmaceutical compositions comprising a crystalline form of Encequidar mesylate, selected from the group consisting of EM1, EM2, EM3 and mixtures thereof together with at least one pharmaceutically acceptable excipient.

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

### Definitions

The term “about” when used in the present application preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

The term “inert solvent” when used in the present application is a solvent that does not react with the reactants or reagents under conditions that cause the chemical reaction indicated to take place.

### EXAMPLES

### Example-1: Preparation of Crystalline Form E1 of Encequidar

A mixture of 2-(2-(4-(2-(6,7-dimethoxy-3,4-dihydroisoquinolin-2(1H)-yl)ethyl)phenyl)-2H-tetrazol-5-yl)-4,5-dimethoxyaniline (22 g) and S-(benzo[d]thiazol-2-yl) 4-oxo-4H-chromene-2-carbothioate (15.9 g) in dichloromethane (440 mL) are stirred at 26° C. for 4 hours. Methanol (28.6 mL) and mixture of acetone (878 mL) and water (46 mL) were added to the above reaction mixture at 26° C. and stirred for 18 hours. The solid was filtered and washed with acetone (110 mL). The wet solid was dried in air tray drier at 45° C. for 3-4 hours to obtain the title compound as solid. XRPD: Crystalline form E1 as depicted in FIG. 1.

### Example-2: Preparation of Crystalline Form EM1 of Encequidar Mesylate Encequidar

(29 g) was dissolved in a mixture of methanol (29 mL)-Chloroform (493 mL) at 26° C. and the solution was filtered to make it particle free. A solution of methane sulphonic acid (2.73 mL) in ethyl acetate (29 mL) was added to the above solution at 26° C. in 40 minutes. Ethyl acetate (116 mL) was added to the reaction mixture at 26° C. in 40 minutes. Ethyl acetate (145 mL) was added to the reaction mixture at 26° C. The reaction mixture was stirred at 26° C. for 16 hours and the solid was filtered. The wet solids are washed with ethyl acetate (87 mL) and dried in air tray drier at 40° C. for 4 hours. The dried solid was combined with mixture of acetone (333 mL) and water (17.5 mL) and stirred at 26° C. for 16 hours. The solid was filtered and washed with acetone (70 mL). The solid was dried in air tray drier at 40° C. for 6 hours. The dried solid was combined with acetone (335 mL) and stirred at 26° C. for 1.5 hours. The solid was filtered and dried in air tray drier at 50° C. for 6 hours to obtain the title compound as solid. XRPD: Crystalline form EM1 as depicted in Figure-2.

The crystalline Form EM1 was subjected to below tabulated stress conditions for 24 hours and found to be stable.

| | Moisture | XRPD |
|---|---|---|
| Stress condition | content (%) | pattern |
| Initial | 6.95 | Form EM1 |
| UV exposed | 6.26 | Form EM1 |
| Open 25° C. | 5.94 | Form EM1 |
| Open 60° C. | 5.5 | Form EM1 |
| Open 40° C./75% RH | 5.71 | Form EM1 |

### Example-3: Preparation of Crystalline Form EM2 of Encequidar Mesylate

A mixture of Encequidar Mesylate (130 mg) and nitromethane (5 mL) was heated to 55° C. and the solution was filtered to make it particle-free. The clear solution was held overnight fat 25° C. in closed container and precipitated solids were separated to obtain the title compound as solid. XRPD: Crystalline form EM2 as depicted in Figure-3.

### Example-4: Preparation of Crystalline Form EM3 of Encequidar Mesylate

Crystalline form EM2 of Encequidar mesylate (100 mg) was dried in an open conical flask at 25° C. for 7 days to obtain the title compound. XRPD: Crystalline form EM3 as depicted in Figure-4.

### Example-5: Preparation of Crystalline Form EM1 of Encequidar Mesylate

A mixture of amorphous (200 mg) and crystalline form EM1 (20 mg) of Encequidar mesylate was suspending in anisole (2 mL) at 25° C. for 22 hours and filtered the solid to obtain title compound. XRPD: Crystalline form EM1.

1. A crystalline Form EM1 of Encequidar mesylate, characterized by X-ray powder diffraction pattern comprising the peaks at 8.78, 10.68, 23.84 and 25.55±0.2° 2θ.

2. The crystalline Form EM1 of Encequidar mesylate according to claim 1, characterized by X-ray powder diffraction pattern as depicted in FIG. 2.

3. A crystalline Form EM2 of Encequidar mesylate, characterized by X-ray powder diffraction pattern comprising the peaks at 16.28, 21.93 and 24.77±0.2° 2θ.

4. The crystalline Form EM2 of Encequidar mesylate according to claim 3, characterized by one or more additional peaks at 12.24, 23.62 and 26.52° 2θ.

5. The crystalline Form EM2 of Encequidar mesylate according to claim 3, characterized by X-ray powder diffraction pattern depicted in FIG. 3.

6. A crystalline Form EM3 of Encequidar mesylate, characterized by X-ray powder diffraction pattern comprising the peaks at 22.85 and 25.91±0.2° 2θ.

7. The crystalline Form EM3 of Encequidar mesylate according to claim 6, characterized by one or more additional peaks at 14.98 and 20.06° 2θ.

8. The crystalline Form EM3 of Encequidar mesylate according to claim 6, characterized by X-ray powder diffraction pattern depicted in FIG. 4.

9. A crystalline Form E1 of Encequidar, characterized by PXRD pattern comprising the peaks at 9.79, 11.00 and 24.07±0.2° 2θ.

10. The crystalline Form E1 of Encequidar according to claim 9, characterized by one or more additional peaks at 19.03, 25.50 and 26.83° 2θ.

11. The crystalline Form E1 of Encequidar according to claim 9, characterized by X-ray powder diffraction pattern depicted in FIG. 1.

12. A process for the preparation of crystalline Form EM1 of Encequidar mesylate according to claim 1, comprising the steps of combining Encequidar mesylate with an inert solvent and isolating crystalline Form EM1.

13. A process for the preparation of crystalline Form EM1 of Encequidar mesylate according to claim 1, comprising the step of treating amorphous Encequidar mesylate with a suitable inert solvent, optionally in the presence of seed crystals of Form EM1.

14. The process of claim 12, wherein the inert solvent is selected from the group consisting of water, ketone solvent, alcohol solvent, ester solvent, nitrile solvent, ether solvent, and mixtures thereof.

15. The process of claim 12, wherein the inert solvent is selected from the group consisting of water, acetone, methanol, ethyl acetate, acetonitrile, anisole and mixtures thereof.

16. A process for the preparation of crystalline Form EM2 of Encequidar mesylate according to claim 3, comprising the steps of combining Encequidar mesylate with nitromethane and isolating crystalline Form EM2.

17. A process for the preparation of crystalline Form EM3 of Encequidar mesylate according to claim 6, comprising the step of drying crystalline Form EM2 of Encequidar mesylate.

18. A process for the preparation of crystalline Form E1 of Encequidar according to claim 9, comprising the steps of combining Encequidar with an inert solvent and isolating crystalline Form E1.

19. The process of claim 18, wherein the inert solvent is selected from the group consisting of water, ketone solvent, alcohol solvent, ester solvent, nitrile solvent and mixtures thereof.

20. The process of claim 18, wherein the inert solvent is selected from the group consisting of water, acetone, methanol, ethyl acetate, acetonitrile and mixtures thereof.

21. A process for the preparation of salt form of Encequidar, comprising the step of converting crystalline Form E1 of Encequidar to salt form of Encequidar.

22. The process of claim 13, wherein the inert solvent is selected from the group consisting of water, ketone solvent, alcohol solvent, ester solvent, nitrile solvent, ether solvent, and mixtures thereof.

23. The process of claim 13, wherein the inert solvent is selected from the group consisting of water, acetone, methanol, ethyl acetate, acetonitrile, anisole and mixtures thereof.
